(19) United States
(12) Patent Application Publication
(10) Pub. No.: US 2009/0163580 A1
(43) Pub. Date: Jun. 25, 2009

(54) Anti-aging composition containing resveratrol and method of administration

(75) Inventors: Michael Todd Yatcilla, Los Angeles, CA (US); Dallas Clouatre, Berkeley, CA (US); Edward Anthony Byrd, San Francisci, CA (US); Amy Addington Fitzpatrick, Kingsport, TN (US)

Correspondence Address: Gabor L. Szekeres;LAW OFFICES OF GABOR L. SZEKERES, P.O. Box 27938, Anaheim, CA, 92809, US

(73) Assignee: Natrol, Inc.

(21) Appl. No.: 12/317,048

(22) Filed: Dec. 18, 2008

Related U.S. Application Data
(60) Provisional application No. 61/009,107, filed on Dec. 24, 2007.

(51) Int. Cl. A61K 31/05; A61K 31/085; A61K 31/353; A61P 25/28; A61P 25/16; A61P 9/00; A61P 35/00; A61P 19/10; A61P 1/16; A61P 1/18

(52) U.S. Cl. 514/456; 514/733; 514/720

(57) ABSTRACT

Formulations and methods of treatment and putative prevention for aging (anti-aging composition) and for diseases or conditions of all reactive oxygen species-dependant illnesses, such as Alzheimer's disease, Parkinson's disease, diabetes mellitus, cardiovascular disease, cancer, hepatitis, and disorders associated with estrogen deficiencies including osteoporosis and breast cancer and for improving athletic performance of humans include resveratrol and two (2) or more of the following features or additional active ingredients: (1) slow release formulation of resveratrol; (2) pterostilbene; (3) quercetin; (4) fisetin, and (5) naringenin. Slow release is defined for the purposes of the present invention as releasing 95% of the active agent or agents in eight (8) hours through normal human gastrointestinal absorption.

### CLAIM OF PRIORITY OF PROVISIONAL APPLICATION

The present application claims the priority of U.S. provisional application 61/009,107 filed on Dec. 24, 2007.

### BACKGROUND OF THE INVENTION

1. Field Of The Invention

The present invention is directed to a composition that contains resveratrol and or closely related compounds and methods of administration for the purpose of preventing, slowing or reversing the biological aging process, improving athletic performance and treating all reactive oxygen species-dependant illnesses, such as Alzheimer's disease, Parkinson's disease, diabetes mellitus, cardiovascular disease, cancer, hepatitis, and treating disorders associated with estrogen deficiencies including osteoporosis and breast cancer, in humans.

2. Brief Description Of The Prior Art

Animal species undergo steady decline in physical and mental function once they reach peak reproductive age (approximately age 20 for humans). This process is greatly accelerated once animals pass reproductive age.

There are several different theories that describe the aging process. However there are very few interventions that describe means of slowing or arresting the aging process. One of the most well-accepted aging interventions is caloric restriction (see Reduced energy intake: the secret to a long and healthy life? Martin B et al., IBS J Sci. September 2007;2(2):35-39.). Caloric restriction has shown the ability to slow the aging process in all animal species tested to date, up to primates. However compliance with caloric restriction is extremely difficult for humans who complain of constant hunger (see One year of caloric restriction in humans: feasibility and effects on body composition and abdominal adipose tissue. (see Racette S B, et al., J Gerontol A Biol Sci Med Sci. September 2006;61(9):943-50.). Hence a dietary or pharmacological agent that mimics the effect of caloric restriction is an unmet need.

There are several biological mechanisms instigated by caloric restriction that must be met by a putative mimic. Among the most prominent and well-researched of these is that caloric restriction activates a family of genes known as “sirtuins” (see Mammalian sirtuins—emerging roles in physiology, aging, and calorie restriction. Haigis M C et al., Genes Dev. Nov. 1, 2006;20(21):2913-21.). These sirtuins turn on a host of biological responses designed to preserve life in the event of emergency (such as starvation). Caloric restriction activates sirtuins, putting the animal into a state of preservation.

There are several plant metabolites that induce sirtuin expression in animals. One of these is the stilbene resveratrol. Resveratrol has shown the ability to induce sirtuin expression in many animals (see Sirtuins in aging and disease. Guarente L. Cold Spring Harb Symp Quant Biol. 2007;72:483-8; Trans-resveratrol: a magical elixir of eternal youth? Orallo F. Curr Med Chem. 2008;15(19):1887-98) including human cells (see Modulation of sirtuins: new targets for antiageing. Pallàs M et al., Recent Patents CNS Drug Discov. January 2008;3(l):61-9). Consistent with the theory of sirtuin expression leading to anti-aging, resveratrol has shown the ability to reduce age-related deterioration in animals (see Resveratrol delays age-related deterioration and mimics transcriptional aspects of dietary restriction without extending life span, Pearson K J et al,. August 2008;8(2): 157-68). However, as used in the prior art resveratrol and other stilbenes have not been found useful in humans (see Walle T et al., High absorption but very low bioavailability of oral resveratrol in humans. Drug Metab Dispos. 2004;32:1377-1382; Wenzel E, Somoza V. Metabolism and bioavailability of trans-resveratrol. Mol Nutr Food Res. 2005;49:472-48 1.). These chemicals are subject to sulfate conjugation in the human intestine (see Resveratrol transport and metabolism by human intestinal Caco-2 cells. Kaldas M I et al., Pharm Pharmacol. March 2003;55(3):307-12.) and metabolized via first-pass detoxification in the liver via glucuronidation. Thus, despite high absorption of the parent and transformed compounds, very little unchanged trans-resveratrol can be found remaining in serum within thirty minutes of ingestion. In well-controlled studies of the prior art, oral doses as high as several grams per day of trans-resveratrol were unable to achieve useful concentrations of trans-resveratrol in serum, which is to say that the resveratrol present in serum was either sulfated or glucuronidated resveratrol rather than trans-resveratrol. The area under the curve (AUC) levels of the metabolites was 23 times that of resveratrol and as much as 77% of ingested resveratrol and its metabolites was excreted within 4 hours. (see Boocock D J et al., Phase I dose escalation pharmacokinetic study in healthy volunteers of resveratrol, a potential cancer chemopreventive agent. Cancer Epidemiol Biomarkers Prev. 2007;16: 1246-1252.).

There are also numerous diseases or conditions in humans that are still in need of effective treatments, or for which treatments may exist but better prevention and or better treatment and or prevention or treatment with less side effects are desired. These include diseases or conditions of all reactive oxygen species-dependant illnesses, such as Alzheimer's disease, Parkinson's disease, diabetes mellitus, cardiovascular disease, cancer, hepatitis, and disorders associated with estrogen deficiencies including osteoporosis and breast cancer. There, thus remains an unmet need for a useful oral formulation of resveratrol and method of treatment with resveratrol by slow release, or in combination with related compounds for acting as anti-aging agent and for treatment of the above-noted diseases or conditions in humans.

### SUMMARY OF THE INVENTION

Resveratrol, pterostilbene, quercetin, fisetin and naringenin are compounds that are known per se and their structural formula is provided below in the detailed description of the Invention.

In accordance with the present invention formulations and methods of treatment and putative prevention are provided for aging (anti-aging composition) and for diseases or conditions of all reactive oxygen species-dependant illnesses, such as Alzheimer's disease, Parkinson's disease, diabetes mellitus, cardiovascular disease, cancer, hepatitis, and disorders associated with estrogen deficiencies including osteoporosis and breast cancer and for improving athletic performance of humans.

The formulations used in accordance with the present invention include resveratrol and two (2) or more of the following features or additional active ingredients: (1) slow release formulation of resveratrol; (2) pterostilbene; (3) quercetin; (4) fisetin, and (5) naringenin. Slow release is defined for the purposes of the present invention as releasing 95% of the active agent or agents in eight (8) hours through normal human gastrointestinal absorption.

In accordance with the foregoing, a slow release formulation of resveratrol combined with any one of the related compounds, namely pterostilbene, quercetin, fisetin, and naringenin is within the scope of the present invention. This formulation or method of treatment may contain or use additional active ingredients, again selected from above-noted four (4) related compounds.

A formulation of resveratrol not specifically designed for slow release but containing or using in a method of treatment two (2) or more of the above-noted four (4) related compounds is also within the scope of the present invention.

### DETAILED DESCRIPTION OF THE INVENTION

Resveratrol is a compound having the formula

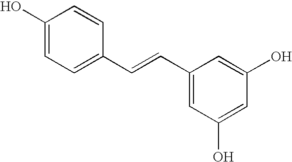

It is also known by the chemical names 3,4′,5′-trihydroxy-trans-stilbene, or 5-[(1E)-2-(4-hydroxyphenyl)ethenyl]-1,3-benzenediol. Commercially available sources of resveratrol include, but are not limited to, an extract from a plant named Polygonum cuspidatum. Extracts of Polygonum cuspidatum standardized to resveratrol content are available commercially from Interhealth USA, 5451 Industrial Way, Benecia, Calif. 94510. This extract usually contains approximately 50% by weight resveratrol. (In the ensuing description all percentages are given by weight.)

Pterostilbene is a compound having the formula

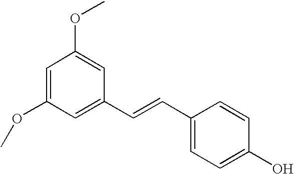

It is also known by the chemical names 4-hydroxy-3′,5!-dimethoxy-trans-stilbene, or 5-[(1E)-2-(4-hydroxyphenyl)ethenyl]-1,3-methoxybenzene. Commercially available sources of pterostilbene include, but are not limited to, an extract from a plant named Pterocarpus marsupium, known in Ayurvedic medicine as “Malabar Kino. This extract usually contains approximately 25% by weight pterostilbene. It is available from Lobsons International, Inc. 14 Highland Avenue, Long Valley, N.J. 07853 under the brand name pTerinol™. A synthesized source of pterostilbene of approximately 95% purity is available from ChromaDex, 10005 Muirlands Blvd, Suite G, First Floor, Irvine, Calif. 92618.

Quercetin is a compound having the formula

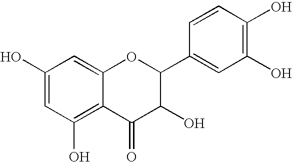

It is also known by the chemical names 3,3′,4′,5′,5,7-pentahydroxyflavone, or 2-(3,4-dihydroxyphenyl)-3,5,7-trihydroxy-4H-chromen-4-one. Quercetin is available commercially as a pure compound from DNP International, 3035 Red Hat Lane, Whittier, Calif. 90601.

Fisetin is a compound having the formula

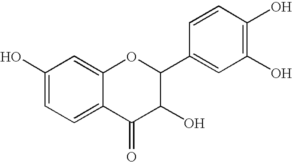

It is also known by the chemical names 3,3′,4′,5′,5,7-pentahydroxyflavone, or 2-(3,4-dihydroxyphenyl)-3,5,7-trihydroxy-4H-chromen-4-one. Fisetin is available commercially as a pure compound from DNP International.

(±) Naringenin is a compound having the formula

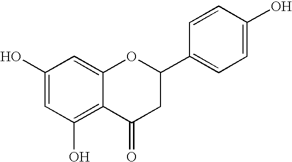

It is also known by the chemical names 4′,5,7-trihydroxyflavone, or 2-(4-hydroxyphenyl)-5,7-dihydroxy-4H-chromen-4-one. Naringenin is available commercially as a pure compound from DNP International.

Although applicant does not wish to be bound by theory, the following “theory” is already mentioned in the prior art section of this application for patent and is summarized here as worthy to be considered in connection with the present invention.

One of the most well-accepted theories for slowing down biological aging is restriction of caloric intake, as such restriction has shown the ability to slow the aging process in all animal species tested to date, up to primates. However, compliance with caloric restriction is extremely difficult for humans, who complain of constant hunger. Nevertheless, there are several biological mechanisms instigated by caloric restriction that could possibly be met by a putative mimic. Among the most prominent and well-researched of these is that caloric restriction activates a family of genes known as “sirtuins”. The sirtuins turn on a host of biological responses designed to preserve life in the event of emergency (such as starvation). Caloric restriction activates sirtuins, putting the animal into a state of preservation. One plant metabolite that induces sirtuin expression is resveratrol. Resveratrol has shown the ability to induce sirtuin expression in many animals and in human cells. Consistent with the theory of sirtuin expression exhibiting anti-aging benefits, resveratrol has shown the ability to reduce age-related deterioration in animals.

In accordance with another theory, resveratrol can act as an antagonist to aryl hydrocarbons, scavenge peroxide and hydroxyl radicals, and chelate copper ions, thereby reducing oxidative stress. It can also act as a suppressor of excessive platelet aggregation.

However, up to the present invention administration of resveratrol to humans has not given satisfactory results in terms of favorably influencing the aging process. The present invention that uses resveratrol in the manner summarized in the “summary” section of this application and is described in detail below overcomes this deficiency in the known art and provides a method of treatment or putative prevention of aging, (anti-aging composition) and for diseases or conditions of all reactive oxygen species-dependant illnesses, such as Alzheimer's disease, Parkinson's disease, diabetes mellitus, cardiovascular disease, cancer, hepatitis, and disorders associated with estrogen deficiencies including osteoporosis and breast cancer and for improving athletic performance of humans.

### Embodiments in Slow Release Form

In accordance with one aspect of the present invention, resveratrol and one or more of the related compounds, namely pterostilbene, quercetin, fisetin, and naringenin (hereinafter “related compounds”) are administered to a human being in a slow release form for the purpose of preventing, slowing down or reversing biological aging and or treating or putatively preventing the other above mentioned diseases and conditions, as well as for the purpose of improving athletic performance. It is believed that daily slow release administration for a prolonged period of time results in resveratrol levels and related compound levels in human plasma that are useful for favorably influencing the biological aging process and the other above-noted conditions or diseases. The slow release is accomplished by providing resveratrol and one or more of the related compounds in a formulation of the type that is normally known to release the active ingredient in a slow process over the course of several hours on the average and preferably in approximately 6 to 8 hours. In accordance with the present invention the formulation is made such that 95 per cent of the active agents are released in eight (8) hours through normal human gastrointestinal absorption, as described above. Such pharmaceutical formulations are known in the art, and a specific example is given below in the description of the preferred embodiments. One of ordinary skill in the art will readily understand that such oral formulations can be in the form of tablet, capsule or other oral release formulations well known in the art.

Thus, in accordance with this aspect of the present invention a daily dose of resveratrol in combination with one or more of the related compounds is administered to an adult human being in a slow release oral formulation. The daily dose, in terms of the resveratrol compound is in the range of 5 to 300 mg (milligram), preferably in the range of 10 to 50 mg, and most preferably is approximately 30 mg.

Again, applicant does not wish to be bound by theory, but it is believed that quercetin, naringenin and fisetin each can act as inhibitors of the cytochrome P450 enzyme. Inhibitory effect on this enzyme slows down the catabolic breakdown and elimination of resveratrol from the human body, and thereby enables the maintenance of resveratrol levels in human serum which have not been made possible in the prior art. These flavones, further, are believed to reduce the activation of glucuronidation and sulfation in the gut lumen, hence to reduce the rate of elimination of compounds, such as of resveratrol, it is not as quickly identified and targeted by liver enzymes and, moreover, it is more effectively assimilated through the intestinal wall.

The daily dose in terms of the pterostilbene compound is in the range of 10 to 500 mg, preferably in the range of 50 to 300 mg, and most preferably is approximately 125 mg. As noted before, pterostilbene is to be administered in combination with resveratrol, preferably but not necessarily included in the same pharmaceutical composition with reservatrol and formulated for slow release.

The daily dose in terms of the quercetin compound is in the range of 50 to 2000 mg, preferably in the range of 100 to 500 mg, and most preferably is approximately 250 mg. As noted before, quercetin is to be administered in combination with resveratrol, preferably but not necessarily included in the same pharmaceutical composition with resveratrol and formulated for slow release.

The daily dose in terms of the naringenin compound is in the range of 50 to 2000 mg, preferably in the range of 100 to 500 mg, and most preferably is approximately 250 mg. As noted before, naringenin is to be administered in combination with resveratrol, preferably but not necessarily included in the same pharmaceutical composition with resveratrol and formulated for slow release.

The daily dose in terms of the fisetin compound is in the range of 50 to 2000 mg, preferably in the range of 100 to 500 mg, and most preferably is approximately 250 mg. As noted before, fisetin is to be administered in combination with resveratrol, preferably but not necessarily included in the same pharmaceutical composition with resveratrol and formulated for slow release.

As a practical matter, some or all of the above noted active ingredients utilized in the formulation and method of treatment of the present invention can be obtained commercially in the form of an extract from a plant, as described above. The presently most preferred slow release formulation of the invented composition includes pterostilbene, resveratrol and quercetin, the first two of which are blended into the tablet as plant extracts.

### Other Embodiments

In accordance with this other aspect of the present invention resveratrol is formulated and used in the methods of anti-aging and other above-described treatments in combination with two of more of the related compounds. For the formulations not specifically adapted for slow release the daily dose of the resveratrol compound is in the range of 10 to 500 mg (milligram), preferably in the range of 50 to 300 mg, and most preferably is approximately 150 mg.

In these formulations the daily dose of pterostilbene compounds is in the range of 5 to 300 mg (milligram), preferably in the range of 10 to 100 mg, and most preferably is approximately 50 mg. The daily dose of quercetin is in the range of 50 to 2000 mg (milligram), preferably in the range of 100 to 1000 mg, and most preferably is approximately 500 mg. The daily dose of fisetin is in the range of 50 to 2000 mg (milligram), preferably in the range of 100 to 1000 mg, and most preferably is approximately 500 mg. The daily dose of naringenin is in the range of 50 to 2000 mg (milligram), preferably in the range of 100 to 1000 mg, and most preferably is approximately 500 mg.

Because these formulations are not specifically designed for slow release resveratrol and the other active agent or agents selected from the related compounds can be formulated with such pharmaceutically acceptable excipients that are commonly used in the art for making tablets, capsules and like oral formulations.

### Presently Preferred Actual Formulations

### Preferred Actual Slow Release Formulation

In the presently most preferred embodiment and best mode for carrying out the present invention with a slow release pharmaceutical composition a tablet is provided that contains the following ingredients per single tablet, and is made in the following manner.

Pterostilbene is included by adding 500 mg per tablet commercially available Pterocarpus marsupium extract that contains 25% pterostilbene, thus providing 125 mg pterostilbene per tablet.

Resveratrol and quercetin are included by adding 480 mg per tablet of a blend (TR blend) that itself contains 60 mg commercially available Polygonium cuspidatum extract containing 50% resveratrol, thus providing 30 mg resveratrol per tablet; 250 mg quercetin powder, and 170 mg hydroxypropylmethylcellulose (HPMC). The Polygonum cuspidatum extract and quercetin powder are blended and then an aqueous solution of HPMC is sprayed onto the blend using a fluid bed granulator of standard design. The resulting granulate dissolves in agitated water over a 6-hour period, releasing the active ingredients by diffusion over 6 hours.

The pharmaceutical composition further contains, dibasic calcium phosphate (200 mg per tablet); stearic acid (35 mg per tablet), croscarmellose sodium (30 mg per tablet); microcrystalline cellulose (150 mg per tablet); silicon dioxide (25 mg tablet); magnesium stearate (15 mg per tablet) and a clear coating blend that adds approximately 10 mg weigh per tablet.

The clear coating contains a standard resin/plasticizer system used for aqueous film coating of tablets. The resin is HPMC and the plasticizer is triacetin.

Except for the features and ingredients described above, the tablet is fabricated in the manner normally used in the art for making a slow release formulation. Those skilled in the art will readily understand that several of the ingredients, except for the active ingredients, are “standard” in the art and can be replaced by equivalents, can be used in different amounts or can be altogether omitted.

The above presently best preferred formulation is also provided here in the form of a Table where some of the ingredients are also mentioned by the trade name known in the art. Weights are in units of milligram (mg).

TABLE I
Slow release tablet formulation.

| | Dose | Weight | |
|---|---|---|---|
| | per | per | Raw Material Amount |
| Ingredient | Day | Tablet | (mg) |
| Pterostilbene | 125 | 125 | As 500 mg of Pterocarpus |
| | | | marsupium extract |
| | | | (standardized to 25% |
| | | | pterostilbene) |
| Resveratrol/Quercitin | 480 | 480 | Granulation containing |
| Slow release granulation | | | 250 mg quercetin, 170 mg |
| | | | HPMC and 60 mg |
| | | | Polygonium cuspidatum |
| | | | extract that has 30 mg |
| | | | resveratrol, granulation |
| | | | designed to deliver |
| | | | nutrients via slow- |
| | | | release |
| Calcium Phosphate Dibasic | 200 | 200 | 200 |
| Stearic Acid FG FCC | 35 | 35 | 35 |
| (Tri-Star 149 tm) | | | |
| Croscarmellose Sodium | 30 | 30 | 30 |
| Microcrystalline | 150 | 150 | 150 |
| Cellulose 102 | | | |
| SIPERNAT 22S | 25 | 25 | 25 |
| (Silicon Dioxide) | | | |
| MAGNESIUM | 15 | 15 | 15 |
| STEARATE | | | |
| Opadry YS-2-7035 | 10 | 10 | 10 |
| (clear coating blend) | | | |

### Other Preferred Actual Formulation

The other presently preferred actual formulation is not specifically designed for slow release. For this reason it can be formulated with such pharmaceutically acceptable excipients that are normally used for making tablets, capsules and other known oral formulations.

The ingredients of the presently preferred actual composition that is not specifically designed for slow release is provided below in Table 2.

TABLE II
Immediate release capsule formulation.

| | Dose | Weight | |
|---|---|---|---|
| | per | per | Raw Material Amount |
| Ingredient | Day | Capsule | (mg) per Capsule |
| Quercetin | 500 | 250 | Granulated powder that |
| | | | contains 95% (by weight) |
| | | | quercitin, approx 264 mg |
| Resveratrol | 150 | 75 | Polygonium cuspidatum |
| | | | extract that contains 50% |
| | | | (by weight) resveratrol, |
| | | | 150 mg |
| Pterostiilbene | 50 | 25 | As 26.3 mg of 95% |
| | | | pterostilbene raw material. |
| Rice Powder (white) | 200 | 200 | |
| Silicon dioxide | 5 | 5 | |
| (SIPERNAT 22S(r)) | | | |
| Magnesium Stearate | 5 | 5 | |
| Clear gelatin CapsuleSize | | | |
| # 00 | | | |

Weights are in units of milligram (mg). This formulation is prepared by methods well known in the art and need not be described here.

What is claimed is:

1. A slow release pharmaceutical composition that comprises a daily unit dose of 5 to 300 mg resveratrol and one or more of the additional compounds selected from the group consisting of pterostilbene in a daily unit dose of 10 to 500 mg, quercetin in a daily unit dose of 50 to 2000 mg, naringenin in a daily unit dose of 50 to 2000 mg and fisetin in a daily unit dose of 50 to 2000 mg.

2. A slow release pharmaceutical composition in accordance with claim 1 that comprises a daily unit dose of 10 to 50 mg resveratrol and one or more of the additional compounds selected from the group consisting of pterostilbene in a daily unit dose of 50 to 300 mg, quercetin in a daily unit dose of 100 to 500 mg, naringenin in a daily unit dose of 100 to 500 mg and fisetin in a daily unit dose of 100 to 500 mg.

3. A slow release pharmaceutical composition in accordance with claim 2 that comprises a daily unit dose of approximately 30 mg resveratrol and one or more of the additional compounds selected from the group consisting of pterostilbene in a daily unit dose of approximately 125 mg, quercetin in a daily unit dose of approximately 250 mg, naringenin in a daily unit dose of approximately 250 mg and fisetin in a daily unit dose of approximately 250 mg.

4. A slow release pharmaceutical composition in accordance with claim 1 comprising a daily unit dose of 5 to 300 mg resveratrol, pterostilbene in a daily unit dose of 10 to 500 mg, and quercetin in a daily unit dose of 50 to 2000 mg.

5. A slow release pharmaceutical composition in accordance with claim 4 comprising a daily unit dose of 10 to 50 mg resveratrol, pterostilbene in a daily unit dose of 50 to 300 mg, and quercetin in a daily unit dose of 100 to 500 mg.

6. A slow release pharmaceutical composition in accordance with claim 2 comprising a daily unit dose of approximately 30 mg resveratrol, pterostilbene in a daily unit dose of approximately 125 mg, and quercetin in a daily unit dose of approximately 250 mg.

7. An immediate-release pharmaceutical composition that comprises a daily unit dose of 10 to 500 mg resveratrol and one or more of the additional compounds selected from the group consisting of pterostilbene in a daily unit dose of 5 to 300 mg, quercetin in a daily unit dose of 50 to 2000 mg, naringenin in a daily unit dose of 50 to 2000 mg and fisetin in a daily unit dose of 50 to 2000 mg.

8. An immediate-release pharmaceutical composition in accordance with claim 7 that comprises a daily unit dose of 50 to 300 mg resveratrol and one or more of the additional compounds selected from the group consisting of pterostilbene in a daily unit dose of 10 to 100 mg, quercetin in a daily unit dose of 100 to 1000 mg, naringenin in a daily unit dose of 100 to 1000 mg and fisetin in a daily unit dose of 100 to 1000 mg.

9. An immediate-release pharmaceutical composition in accordance with claim 7 comprising a daily unit dose of 50 to 300 mg resveratrol, pterostilbene in a daily unit dose of 10 to 100 mg, and quercetin in a daily unit dose of 100 to 1000 mg.

10. An immediate-release pharmaceutical composition in accordance with claim 7 comprising a daily unit dose of approximately 150 mg resveratrol, pterostilbene in a daily unit dose of approximately 50 mg, and quercetin in a daily unit dose of 500 mg.

11. A method of administering to a human being for the purpose of preventing, reversing or slowing down the biological aging process, treating reactive oxygen species-dependant illnesses, disorders associated with estrogen deficiencies and for improving athletic performance a slow-release pharmaceutical composition in accordance with claim 1.

12. A method in accordance with claim 11 of administering to a human being for the purpose of preventing, reversing or slowing down the biological aging process, treating reactive oxygen species-dependant illnesses, disorders associated with estrogen deficiencies and for improving athletic performance a pharmaceutical composition comprising: a daily dose of 10 to 50 mg resveratrol and one or more of the additional compounds selected from the group consisting of pterostilbene in a daily dose of 50 to 300 mg, quercetin in a daily dose of 100 to 500 mg, naringenin in a daily dose of 100 to 500 mg and fisetin in a daily dose of 100 to 500 mg.

13. A method in accordance with claim 11 of administering to a human being for the purpose of preventing, reversing or slowing down the biological aging process, treating reactive oxygen species-dependant illnesses, disorders associated with estrogen deficiencies and for improving athletic performance a pharmaceutical composition comprising:
a daily dose of 5 to 300 mg resveratrol, pterostilbene in a daily dose of 10 to 500 mg, and quercetin in a daily unit dose of 50 to 2000 mg.

14. A method in accordance with claim 13 of administering to a human being for the purpose of preventing, reversing or slowing down the biological aging process, treating reactive oxygen species-dependant illnesses, disorders associated with estrogen deficiencies and for improving athletic performance a pharmaceutical composition comprising:
a daily unit dose of approximately 30 mg resveratrol, pterostilbene in a daily unit dose of approximately 125 mg, and quercetin in a daily unit dose of 250 mg.

15. A method of administering to a human being for the purpose of preventing, reversing or slowing down the biological aging process, treating reactive oxygen species-dependant illnesses, disorders associated with estrogen deficiencies and for improving athletic performance an immediate-release pharmaceutical composition in accordance with claim 7.

16. A method in accordance with claim 15 of administering to a human being for the purpose of preventing, reversing or slowing down the biological aging process, treating reactive oxygen species-dependant illnesses, disorders associated with estrogen deficiencies and for improving athletic performance a pharmaceutical composition comprising:
a daily dose of 50 to 300 mg resveratrol and one or more of the additional compounds selected from the group consisting of pterostilbene in a daily dose of 10 to 100 mg, quercetin in a daily dose of 100 to 1000 mg, naringenin in a daily dose of 100 to 1000 mg and fisetin in a daily dose of 100 to 1000 mg.

17. A method in accordance with claim 15 of administering to a human being for the purpose of preventing, reversing or slowing down the biological aging process, treating reactive oxygen species-dependant illnesses, disorders associated with estrogen deficiencies and for improving athletic performance a pharmaceutical composition comprising:
a daily unit dose of 50 to 300 mg resveratrol, pterostilbene in a daily unit dose of 10 to 100 mg, and quercetin in a daily unit dose of 100 to 1000 mg.

18. A method in accordance with claim 17 of administering to a human being for the purpose of preventing, reversing or slowing down the biological aging process, treating reactive oxygen species-dependant illnesses, disorders associated with estrogen deficiencies and for improving athletic performance a pharmaceutical composition comprising:
a daily dose of approximately 150 mg resveratrol, pterostilbene in a daily dose of approximately 50 mg, and quercetin in a daily dose of 500 mg.

19. A method in accordance with claim 11 comprising administering the pharmaceutical composition in daily dose as set forth in claim 11 for the purpose of preventing, reversing or slowing down the biological aging process.

20. A method in accordance with claim 15 comprising administering the pharmaceutical composition in daily dose as set forth in claim 15 for the purpose of preventing, reversing or slowing down the biological aging process.
